(19) United States
(12) Patent Application Publication
(10) Pub. No.: US 2025/0359794 A1
(43) Pub. Date: Nov. 27, 2025

(54) Device for Detecting Magnetic Signals Generated by a Beating Heart

(71) Applicant: Robert Bosch GmbH, Stuttgart (DE)

(72) Inventors: Florian Dolde, Stuttgart (DE); Frederik Schaal, Reutlingen (DE); Florian Krist, Stuttgart (DE); Riccardo Cipolletti, Geislingen An Der Steige (DE); Eckhard Wehrse, Stuttgart (DE)

(21) Appl. No.: 19/109,007

(22) PCT Filed: Aug. 18, 2023
(86) PCT No.: PCT/EP2023/072816

§ 371 Date: Mar. 5, 2025

(30) Foreign Application Priority Data
Sep. 9, 2022 (DE) 10 2022 209 424.7

(51) Int. Cl. A61B 5/243; A61B 5/00; G01R 33/26

(52) U.S. Cl. CPC A61B 5/243; A61B 5/70; G01R 33/26; A61B 2562/0223

(57) ABSTRACT

A device is for detecting magnetic signals generated by a beating heart. The device includes a support body having a contact surface, and an arrangement of at least two nitrogen-vacancy centers, NV, magnetometer units. The arrangement is embedded in the support body. The support body is configured to receive a user sitting or lying on the contact surface.

The present invention relates to a device for detecting magnetic signals generated by a beating heart.

### BACKGROUND OF THE INVENTION

Optically pumped quantum sensors or those based on NV centers in diamond are particularly suitable for measuring very small magnetic field strengths. DE 10 2022 204 526.2 describes a magnetometer that utilizes optically pumped and optically detected magnetic resonance (ODMR). This utilizes the fact that the energy levels of certain spin states of unpaired electrons split under the influence of an external magnetic field, the so-called Zeeman effect. The splitting of the energy levels results in changed transitions during relaxation from excited states, which can then be measured, for example, by optical excitation and frequency-dependent detection of the resulting fluorescence radiation or by observation of optical properties such as the absorption of light. From the measured optical parameters, the magnetic field strength may then in turn be inferred.

### DISCLOSURE OF THE INVENTION

A device for detecting magnetic signals generated by a beating heart is proposed according to the invention, having the features of claim 1. Advantageous configurations are the subject matter of the dependent claims and the following description.

A magnetocardiogram (abbreviated MCG) is the recording and visualization of the magnetic field of the heart, which is caused by the electrophysiological activity of the heart muscle cells. The invention presents a contactless, passive option for long-term monitoring of the human heart at high resolution. This is realized by a geometric arrangement of nitrogen-vacancy magnetometers (so-called NV magnetometers).

In particular, a device for detecting magnetic signals generated by a beating heart is presented, which comprises a base body having a contact surface and an arrangement of at least two NV magnetometer units, wherein said arrangement is embedded in said support body, wherein said support body is adapted to receive a user sitting or lying on said contact surface. Such a device can also be called a magnetocardiograph.

One particular advantage of the NV sensor system is its size, especially the sensor medium. For the application, the active measuring volume should be small compared to the object to be measured (heart), otherwise the surface coverage over large parts of the signal will cause integration and thus the signal may disappear because the integral is zero. The smaller the active measuring volume compared to the heart, the better the signal detection. NV sensors have a very small active sensor volume. This small size also allows the sensors to be used in a geometric arrangement. In particular, very high-resolution arrangements are possible due to the very small active sensor volume.

This also allows for easy integration into textiles or other everyday objects, wherein numerous options are considered. In one embodiment, the support body is a cushion, a mattress, a couch, a mat, a bed, a seat (such as a car seat) or a chair; it can also be integrated into, for example, toppers, underlays, covers, slatted frames, bed frames, duvets, pillows, side sleeper pillows, etc.

Diamond NV magnetometers rely on the readout of the magnetic resonance of special defect centers in diamond, in particular of nitrogen vacancies (NV) that occur as impurities in the carbon lattice of diamond or can be introduced deliberately. When the NV center is optically excited in the ground state, e.g. by irradiating a pump laser beam of a suitable wavelength (in this case in the green wavelength range, e.g. at 532 nm for off-resonance excitation), the electrons are excited from the ground state to the triplet excited state and relax by emitting fluorescent light in the red wavelength range at 650-800 nm (637 nm=zero phonon line). Since non-spin-conserving transitions from the spin state with spin quantum number mₛ=±1 are more likely, continuous excitation pumping ensures that the NV centers are mostly hyperpolarized in the spin state mₛ=0.

There is an energy difference between the mₛ=0 and mₛ=±1 spin states in the ground state, which in this case is about 2.87 GHz. Thus, if microwave radiation is radiated into the diamond in addition to the optical excitation, there is a dip in the red fluorescence at this resonance frequency of 2.87 GHz, because the spin-polarized electrons are lifted by the microwave field from mₛ=0 to the mₛ=±1 ground state and excited from there by the pump light to the mₛ=±1 excited state. From there, however, primarily non-radiative transitions and weak infrared fluorescence transitions occur via the singlet state, and the fluorescence in the red range ceases.

If an external magnetic field is present, the so-called Zeeman effect leads to the splitting of the otherwise energetically equal mₛ=±1 triplet levels into energetically equidistant Zeeman levels. When plotting fluorescence against a frequency spectrum of microwave excitation, two dips appear in the fluorescence spectrum, the frequency spacing of which is proportional to the magnetic field strength of the external magnetic field. The magnetic field sensitivity is defined primarily by the minimum resolvable frequency shift and can reach 1 pT/√Hz or less. Since the NV center in the single-crystal diamond has four possibilities for arranging itself in the crystal lattice, the NV centers present in the crystal react to the external magnetic field to different degrees depending on their position in the crystal when a magnetic field is applied. As a result, four pairs of fluorescence minima may appear in the spectrum, from the shape and position of which both the magnetic field strength as an amount and the direction of the external magnetic field are clearly determinable.

In order to enable vectorial magnetic field measurements, the device has a feature that creates a substantially homogenous bias magnetic field in the area of the magnetometer units or their sensor media. The mechanism can also be integrated into the support body. It may be a Helmholtz coil arrangement, wherein at least the sensor medium of the at least two NV magnetometer units is arranged within the Helmholtz coil arrangement. It can also be other devices such as a simple coil, an elongated coil, permanent magnet solutions such as in a Hallbach array, etc.

At a distance of a few centimeters, cardiac signals have a magnetic signature with an amplitude of (only) 1 to 2-digit picotesla (pT), whereas, for example, the earth's magnetic field in Central Europe is about 50 μT (microtesla), i.e. it is stronger by a factor of 106. However, even such small field strengths can be resolved with high precision over a long period of time using the proposed technology. For example, magnetic shielding or a gradiometer circuit can be used for this purpose.

The high-resolution detection of the exact heart signal allows a variety of diseases to be detected, such as permanent atrial fibrillation and paroxysmal atrial fibrillation. This can help to prevent a heart attack and subsequentstroke (especially after an undetected heart attack). Furthermore, the invention is suitable for the early detection of an S-T elevation myocardial infarction, a different type of elevationmyocardial infarction, a pulmonary embolism, an AV nodal reentry tachycardia, ventricular premature beats, but also very rare pathogenic diseases such as arrhythmogenic right ventricular tachycardia, which can otherwise only be detected by gene sequencing.

Only a precise resolution of the heart signal makes it possible to detect these diseases. In the case of heavily noisy or poorly resolved signals, the shifts of the various PQRST complexes of the heart against each other or over time, fluctuations in their amplitude, deformations or small disturbances are not detectable. These criteria are important factors, however, as the above-mentioned problems can lead to complexes being mixed up (e.g. interpretation of elevated and shifted T wave as R wave, which, however, often occurs in a “healthy” heart) and to false alarms.

When using a gradiometer circuit of at least two NV magnetometer units, one magnetometer unit is always further away from the center (as a relatively weak magnetic field source) than another magnetometer unit. The gradiometer configuration, i.e. essentially (vectorial) subtraction of the measured, means that the magnetic field gradient approximately corresponds to the field emanating from the weak source, while significantly stronger background fields (which are essentially the same in both magnetometer units) are eliminated. This eliminates the need for magnetic shielding, making it possible to measure magnetic fields in everyday environments. Accordingly, the invention is particularly suitable for the unshielded measurement of weak magnetic fields. Technical details of gradiometer solutions that can also be used in the context of the present invention are disclosed in DE 102022201690.4 and are to be included here.

The NV sensor technology is integrated in such a way that it is not noticeable or disruptive. In one embodiment, the support body comprises resilient material between the arrangement and the contact surface. By selecting special materials in terms of thermal conductivity, elasticity, hardness, etc., a certain degree of temperature control or pressure control can also be achieved so that the LV units are not damaged and people are not injured.

In one embodiment, at least one structure made of a material with high magnetic permeability >>1, e.g. greater than 10, 100 or 1000, in particular ferromagnetic material, e.g. containing iron, cobalt, nickel, is provided on a side of the arrangement facing away from the contact surface and/or in an (additional) contact body. This can be used for field guidance or shielding (e.g. from other magnetic fields or microwaves). In particular, the structure may comprise a layer, e.g. a plate or a film, a lattice structure, e.g. a mesh, etc. If the structure is intended on a side of the arrangement facing away from the contact surface, it can be arranged under the support body or embedded in the support body. The contact body can be a topper or a duvet.

In one embodiment, the device is configured to detect magnetic field strength and field direction using each of the at least two NV magnetometer units. Another advantage of the NV sensor system is the directional or vector information. In contrast to other technologies, this is intrinsic to NV sensor technology. This means that neither interference nor unfavorable projections need to be introduced by modulation techniques, nor do several separate sensors have to be used. This means that the vector and gradiometry information is available for the exact same location (diamond size, i.e. single-digit mm\hat{ }3 and below), and not separated by a few centimeters to many centimeters as with other technologies. NV magnetometer units that can determine not only the field strength but also the direction of the magnetic field enable improved suppression of a background field and thus better detection of signals that are heavily superimposed by interference signals.

In one embodiment, the device has a signal processing unit to which the at least two NV magnetometer units are connected, wherein the device is configured to determine an effective magnetic field strength and/or an effective magnetic field direction as the difference in the magnetic field strength or field direction detected by means of the at least two NV magnetometer units by means of the signal processing unit. Both a wireless and a wired connection between the sensor technology and the signal processing unit are planned.

For the application, a sampling rate is required that is higher than the heart signal in order to resolve it, in particular greater than 50 Hz. A range of 200 Hz to 400 Hz is considered particularly beneficial. Higher is always better for the resolution, but it increases the requirements for sensitivity.

Further advantages and embodiments of the invention will emerge from the description and the accompanying drawings.

The invention is shown schematically in the drawings on the basis of exemplary embodiments and is described hereinafter with reference to the drawings.

### BRIEF DESCRIPTION OF THE DRAWINGS

FIG. 1 shows a schematic block diagram of the essential components of an NV center magnetometer as it can be used in the context of the invention.

FIG. 2 shows in various figures a) through c) in each case in a schematic block view possible arrangements of NV magnetometer units of a device for detecting magnetic signals according to an embodiment.

FIG. 3 schematically shows a side view of a user on a support body according to an embodiment of the invention.

FIG. 4 schematically shows possible designs of support bodies according to embodiments of the invention in three side views a) through c).

FIG. 5 schematically shows possible designs of support bodies in accordance with embodiments of the invention, in a side view.

FIG. 6 schematically shows possible designs of support bodies according to embodiments of the invention in four side views a) through d).

FIG. 7 schematically shows in six plan views a) through f) possible configurations of arrangements with one or more NV magnetometer units according to embodiments of the invention.

FIG. 8 schematically shows in four side views a) through d) possible configurations of devices with different support bodies with one or more arrangements with NV magnetometer units according to embodiments of the invention.

FIG. 9 schematically shows in two side views a) and b) possible configurations of devices with several arrangements of NV magnetometer units and a signal processing unit according to embodiments of the invention.

FIG. 10 schematically shows a side view of an arrangement of a device with several arrays of NV magnetometer units, a signal processing unit and an auxiliary device according to one embodiment of the invention.

### EMBODIMENT(S) OF THE INVENTION

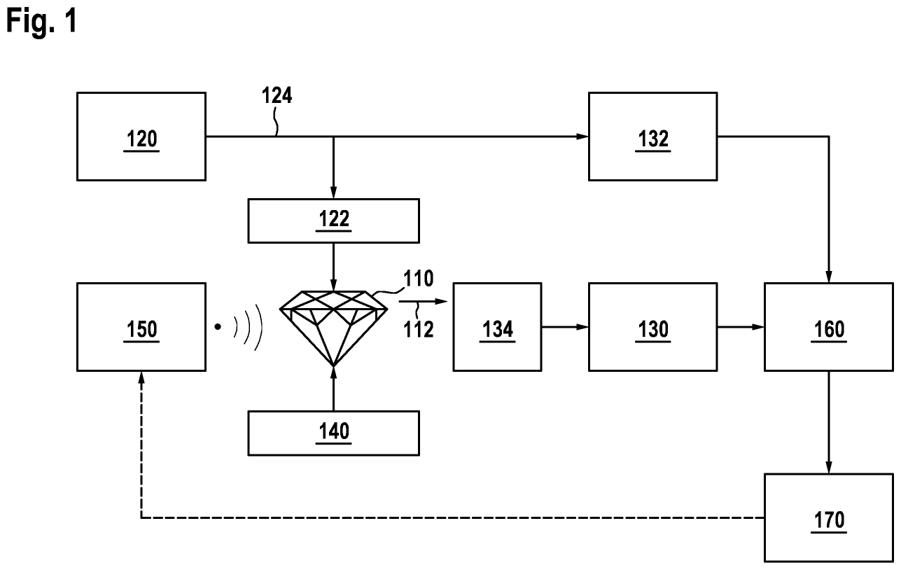

FIG. 1 schematically shows the essential components of a NV-center magnetometer. Initially, a 110 diamond with nitrogen vacancies (NV) is used as the sensor medium. Optical excitation of the NV centers can be achieved using a suitable light source 120 such as a pump laser. A frequency-doubled Nd:YAG laser or semiconductor laser in the green range of about 510-532 nm, e.g. at 532 nm for off-resonance excitation, is suitable here. Alternatively, LEDs in suitable wavelength ranges can also be used. Depending on the arrangement, the light from the light source 120 can be irradiated into the diamond 110 via suitable optical elements 122 such as mirrors, beam splitters, focusing optics such as lenses and, if necessary, via fiber optic elements. Furthermore, the excitation light can be irradiated continuously or pulsed by the laser, so that, for example, time windows for interference-free fluorescence light measurement are kept free.

Furthermore, the magnetometer can include a microwave source 150 that is able to generate an electromagnetic field in the sensor medium, i.e. in the area of the NV centers of the diamond 110, over a bandwidth that covers the desired resonance frequency. A microwave resonator structure can be used to distribute the generated microwaves homogeneously throughout the volume of the measuring area in the diamond. The resonator structure or microwave source 150 is tuned to the frequency of the electron spin resonances. To enable vector magnetometry, an additional static bias magnetic field 140 is generated. This makes the measurement intrinsically vector-based. To do this, different spatial directions are used in the crystal structure. A Helmholtz coil is suitable for generating such a magnetic field 140, in which a pair of coils can be used to generate a largely homogeneous magnetic field in a confined area.

The fluorescent light 112 emerging from the diamond 110 can in turn be directed via suitable optical elements 134, such as optical filters, beam splitters, lenses, and/or fiber-optic elements, to a first photodetector 130 that is sensitive at least in the range of the fluorescence wavelength. The first photodetector 130 can also be arranged directly on the diamond 110. A second photodetector 132 is arranged so that it can detect at least some of the excitation light from the light source 120, which can be decoupled, for example, by a beam splitter, a filter or a partially permeable element. This detector signal 132 of the excitation light can be used as a reference signal, for example, to eliminate background signals and emphasize the resonance signal of interest by modulating the excitation light using a lock-in amplifier. Additionally or alternatively, this reference signal can be used to take fluctuations in the excitation light into account. Corresponding circuits 160 such as a preamplifier, a logarithmic amplifier, a lock-in amplifier, signal filters or others are therefore provided to receive the signals from the first and second photodetectors and to preprocess the signals in a suitable way for further evaluation. Finally, a signal processing unit 170 can be used to evaluate the preprocessed fluorescence signal, e.g. with a suitable microcontroller or processor, in order to obtain the desired parameters of the detected magnetic field from the signal, in particular the magnetic field strength and the direction of the magnetic field.

It is understood that such a device may also include other, not shown, units, such as communication units or interfaces for outputting the measurement results. Such a device can also be advantageously integrated into an ASIC or FPGA.

To be applicable in an everyday environment, magnetic fields that do not originate from the desired weak sources should be eliminated from the measurement as far as possible, in particular the earth's magnetic field in the range of 10⁻⁵ Tesla (a few microtesla). In contrast, the magnetic fields of the heart are in the range of 10-100 times 10⁻¹² Tesla (picotesla).

The elimination of the background magnetic fields can be achieved by shielding or by a gradiometer arrangement in the magnetic field measurement according to exemplary embodiments. Gradiometers are generally referred to as sensor units that are capable of detecting not only the field strength, but also the gradient of the field.

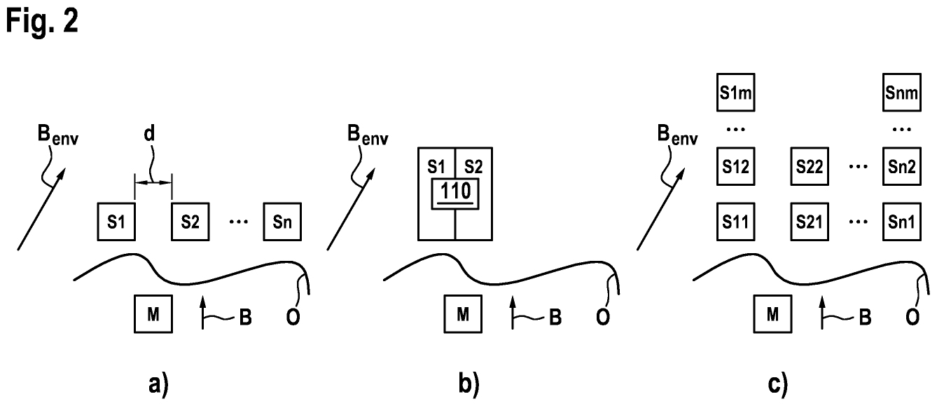

For this purpose, at least two individual magnetometer units can be used, which are arranged at different locations. As an example, a sensor unit that uses two or more NV center magnetometers in a gradiometer arrangement is described below in connection with FIG. 2.

FIG. 2 shows in various figures a) through c) possible geometric arrangements of NV magnetometer units of a device for detecting magnetic signals according to an embodiment. Figure a) shows a side view of an arrangement of at least two NV magnetometer units S1, S2, . . . , Sn in an arbitrary arrangement to each other in a plane (perpendicular to the drawing plane, i.e. only the first row is visible). Figure b) shows a side view of two NV magnetometer units, S1 and S2, whose sensor media are sections of the same diamond crystal 110. Figure c) shows a side view of a number (n times m) of NV magnetometer units S11, S21, . . . , Sn1, S12, S22, . . . , Sn2, S1m, . . . , Snm in an arbitrary three-dimensional arrangement. Additional layers are added behind the drawing layer, so that a kind of cubic lattice is formed overall. In this case, at least one NV magnetometer unit (not shown), which is located, for example, in one of the back layers, is not arranged in the plane (drawing plane) in which the other NV magnetometer units S11, S21, . . . , Sn1, S12, S22, . . . , Sn2, S1m, . . . , Snm are arranged.

Furthermore, M denotes a signal source, here a heart, and O denotes an optional surface (in particular, body skin), which limits the accessibility to or reachability of the magnetic field source M.

In embodiments of the invention, two NV magnetometer units can always form a gradiometer, wherein—depending on the number of NV magnetometer units—a total of several gradiometers are formed and detect the signal of interest. An effective measurement signal can then be formed from this, in particular by the signal processing unit, for example by averaging, summation, etc.

A distance d between two NV magnetometer units S1, S2, . . . or, more precisely, their sensor media corresponds to the distance between the locations where simultaneous magnetic field measurements are taken. As long as the distance between the measuring points is relatively small, it can be assumed that the strength of an additional background magnetic field Bₑₙᵥ is approximately the same at both points. In contrast, the weak magnetic field B of interest will decrease significantly with increasing distance from the magnetic field source M.

Thus, by placing two NV magnetometer units at different distances from the source or heart, the background field can be eliminated by forming a difference between the detected sensor values and the small magnetic field of interest or its gradient can be extracted: Since the magnetic field weakens with the square of the distance, the largest magnetic field change is detected by the NV magnetometer units near the source. For this purpose, for example, two NV magnetometer units can be arranged one above the other in an axial gradiometer configuration, so that each NV magnetometer unit of a first layer with an underlying NV magnetometer unit of a second, underlying layer forms a gradiometer. The background field can also be determined by means of a further NV magnetometer unit at a large distance, e.g. at least 1 m, from the two NV magnetometer units.

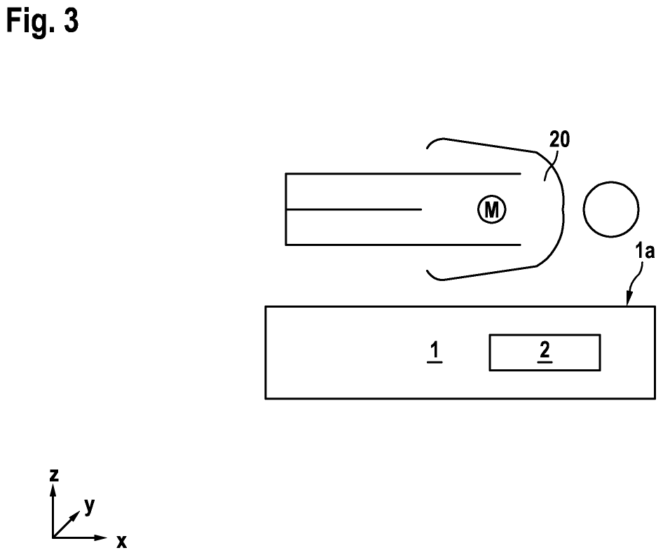

FIGS. 3 through 10 schematically show possible embodiments of the invention and are described in general terms below. Identical elements are labeled with the same reference signs and are not described multiple times.

A device 2 for detecting magnetic signals is shown in each case, which has a support body 1 with a contact surface la and at least one arrangement 3 of at least two nitrogen-vacancy, NV, centers magnetometer units 4, wherein the at least one arrangement 3 is embedded in the support body 1. The support body is designed to accommodate one user 20, either sitting or lying on the contact surface. The device 2 is used to detect magnetic signals generated by a beating heart (M), but it can in principle detect all magnetic signals, in particular bio-signals, i.e. those that emanate from living beings. To illustrate this, the figures each have a coordinate system in the top left corner, wherein the drawing plane represents the x-z plane and the y-axis runs into the drawing plane.

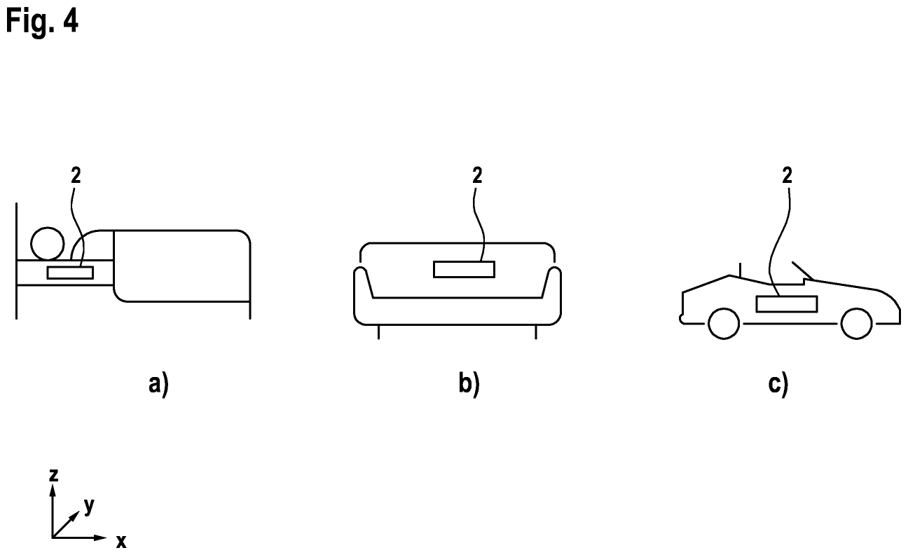

FIG. 3 shows a mattress as a support body 1, FIG. 4a) shows a mattress in a bed, FIG. 4b) shows a sofa and FIG. 4c) shows a car seat.

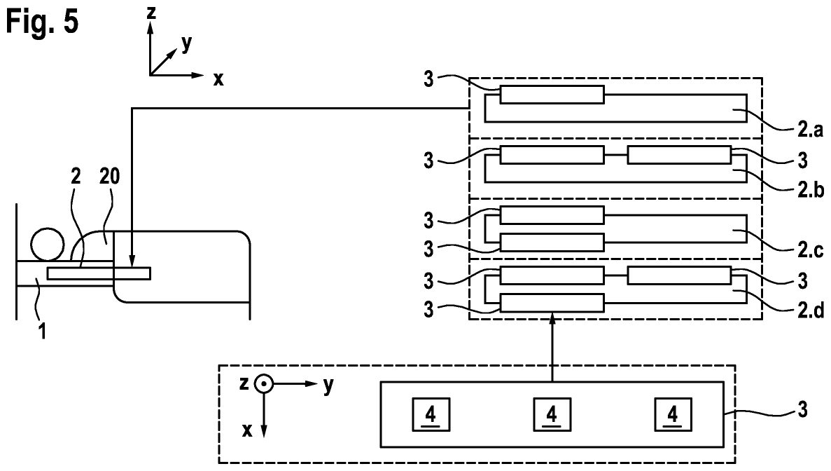

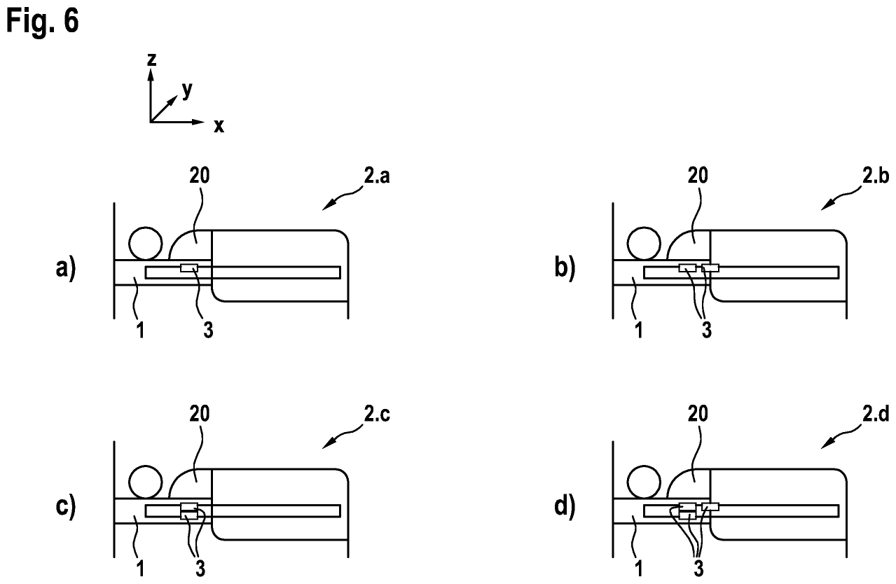

FIG. 5 shows a schematic side view of an extended device 2 in a mattress of a bed with a user, as it can be used for long-term monitoring, in particular of magnetic heart signals. On the right, FIG. 5 and FIG. 6 show various options 2.a through 2.d for how one or more arrangements 3 of NV magnetometer units 4 can be arranged in a device 2. A device can have one arrangement (variant 2.a) or more than one arrangement (variants 2.b through 2.d). The arrangements can also be arranged in a specific geometric arrangement, for example in a line (1D), plane (2D) or distributed in space (3D). At the bottom right of FIG. 5, a schematic of an arrangement 3 with several NV magnetometer units 4 is shown in a plan view, which are themselves also arranged in a geometric arrangement, here as a line. The NV magnetometer units 4 of an arrangement 3 can themselves also be arranged in a certain geometric arrangement, for example in a line (1D), plane (2D) or distributed in space (3D), as already explained in connection with FIG. 2. As explained, two NV magnetometer units can always form a gradiometer, wherein—depending on the number of NV magnetometer units—a total of several gradiometers can be formed to detect the signal of interest. An effective measurement signal can then be formed from this, in particular by the signal processing unit, for example by averaging, summation, etc.

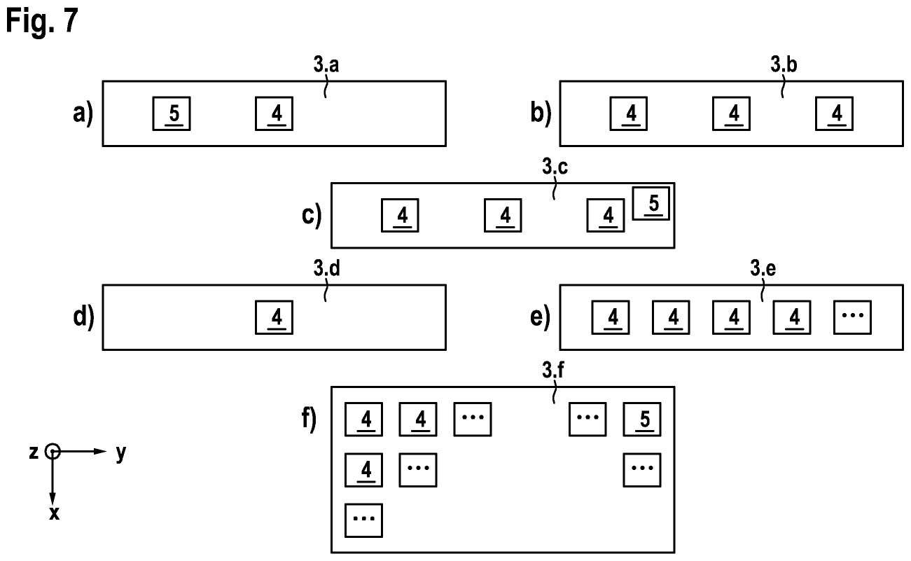

FIG. 7 shows in schematic top view in different views a) through f) variants 3.a through 3.f of arrangements 3 with one or more NV magnetometer units 4, each with no, one or more additional sensors 5. The sensors 5 can be, in particular, pressure sensors, pulse oximeters, temperature sensors, etc. The NV magnetometer units 4 and/or the sensors 5 of an arrangement 3 can be arranged in a specific geometric arrangement, for example in a line (1D), plane (2D) or distributed in space (3D), as already explained in connection with FIG. 2 or 5.

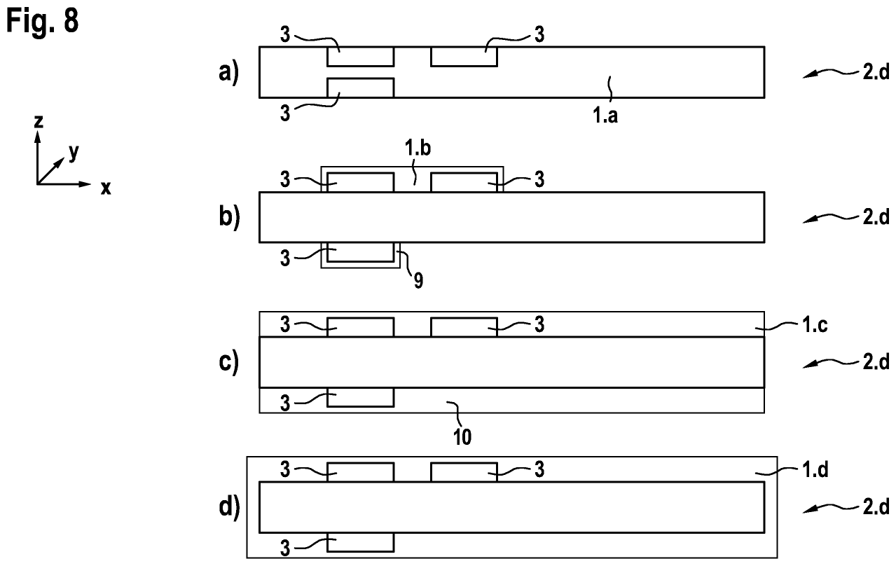

FIG. 8 shows in four side views a) through d) various variants 2.d of a device 2 with two arrangements 3 in the area of an upper side and one arrangement 3 in the area of a lower side of a support body. In version a), the three arrangements 3 are embedded in a mattress 1.a as a support body. In version b), two arrangements 3 are embedded in a cushion 1.b as a support body. In addition, an arrangement is provided under the mattress, e.g. in a base plate 9. In version c), two arrangements 3 are embedded in a topper 1.c as a support body. In addition, an arrangement is provided under the mattress, e.g. in a slatted frame 10. In variant d), the three arrangements 3 are embedded in a mattress cover 1.d as a support body. Various mechanisms can be used for this purpose, e.g. layers, e.g. foam, e.g. covers, e.g. various wrapping materials, e.g. materials to protect the electronics but also for shielding and increasing comfort.

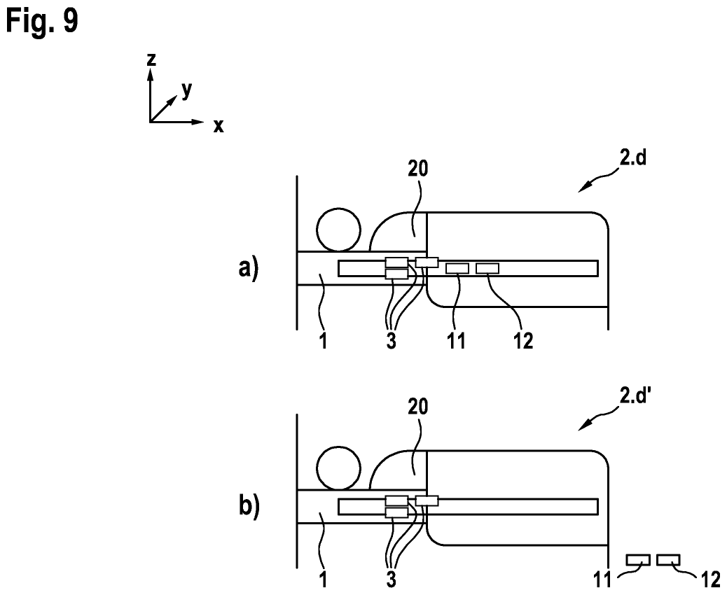

FIG. 9 shows in two side views a) and b) various variants 2.d, 2.d′ of a device 2 with two arrangements 3 in the area of an upper side and an arrangement 3 in the area of a lower side of a support body 1, in particular of a mattress. Furthermore, the device has a signal processing unit 11 to which the NV magnetometer units of the arrangements 3 are connected in order to determine an effective magnetic field strength and/or field direction. Furthermore, a communication unit 12 can be provided to connect the device 2 to other devices such as a PC, tablet PC, smartphone for input and output and operation. The communication unit 12 can, for example, have wired and/or wireless interfaces. In variant 2.d, the signal processing unit 11 and communication unit 12 are also integrated into the support body, and in variant 2.d′, they are arranged outside the support body.

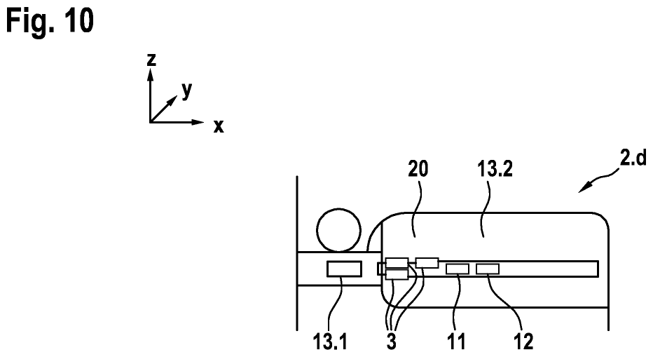

FIG. 10 schematically shows a side view of an arrangement of a device 2.d with several arrangements 3 with NV magnetometer units, a signal processing unit 11, a communication unit 12 and two variants of auxiliary devices 13.1, 13.2 according to embodiments of the invention.

The auxiliary device 13.1, 13.2 can fulfill at least one function, selected from a function for dissipating waste heat, for heat shielding, for heat conduction, for magnetic field compensation (e.g. actively by coils), for (electro-)magnetic shielding, for protection against moisture and for increasing comfort (use of certain packaging and composite materials to make sleeping pleasant and comfortable). The auxiliary device 13.1, 13.2 can have a structure, for example a network, made of a ferromagnetic material with high magnetic permeability, e.g. greater than 100.

The auxiliary device 13.1 can also be embedded in the support body. It can also be embedded under the support body, e.g. in a bed frame or slatted frame, or in a contact body 13.2 on the user 20, e.g. in the form of a cover.

1. A device for detecting magnetic signals generated by a beating heart, comprising:
a support body with a contact surface; and
an arrangement of at least two nitrogen-vacancy centers, NV, magnetometer units, the arrangement embedded in the support body,
wherein the support body is configured to accommodate a user sitting or lying on the contact surface.

2. The device according to claim 1, wherein the support body has elastic material between the arrangement and the contact surface.

3. The device according to claim 1, wherein the support body is a cushion, a mattress, a couch, a mat, a bed, a seat, or a chair.

4. The device according to claim 1, further comprising:
a structure made of a material with a magnetic permeability greater than 1 on a side of the arrangement facing away from the contact surface, and/or a contact body containing the structure.

5. The device according to claim 1, wherein the device is configured to detect a magnetic field strength and a field direction using each of the at least two NV magnetometer units.

6. The device according to claim 1, further comprising:
a signal processing unit to which the at least two NV magnetometer units are connected,
wherein the device is configured to determine, using the signal processing unit, an effective magnetic field strength and/or field direction as a difference of magnetic field strengths and/or field directions detected using the at least two NV magnetometer units.

7. The device according to claim 1, wherein the arrangement is a two-dimensional arrangement in which the at least two NV magnetometer units are arranged in a plane.

8. The device according to claim 1, wherein:
the at least two NV magnetometer units comprise at least four NV magnetometer units, and
the arrangement is a three-dimensional arrangement in which at least one of the at least four NV magnetometer units is not arranged in a plane in which at least three others of the at least four NV magnetometer units are arranged.

9. The device according to claim 1, wherein:
each of at least two NV magnetometer units has, as a sensor medium, a diamond crystal, or a section of a diamond crystal having nitrogen-vacancy centers, and
the device is configured to detect a magnetic field strength and/or a field direction by reading a spin resonance dependent on the magnetic field strength in the sensor medium.

10. The device according to claim 9, further comprising:
at least one excitation light source configured to radiate light into the sensor medium;
at least one microwave source configured to generate a resonant field in the sensor medium; and
at least one photodetector configured to detect resonance-dependent fluorescent light from the sensor medium.

11. The device according to claim 10, wherein a same excitation light source and/or a same microwave source are associated with the at least two NV magnetometer units.

12. The device according to claim 9, wherein the sensor medium of the at least two NV magnetometer units each has a portion of a the same diamond crystal.

13. The device according to claim 9, wherein a distance between the sensor media of the at least two NV magnetometer units is from 1 to 30 millimeters.

14. The device according to claim 1, further comprising:
a further NV magnetometer unit at a distance of least 1 m from the at least two NV magnetometer units.
